(19) United States
(12) Patent Application Publication
(10) Pub. No.: US 2026/0216133 A1
(43) Pub. Date: Jul. 30, 2026

(54) AEROSOL COMPRISING 5-METHOXY-N,N-DIMETHYLTRYPTAMINE

(71) Applicant: GH Research Ireland Limited, Dublin 2 (IE)

(72) Inventor: Theis Terwey, Berlin (DE)

(73) Assignee: GH Research Ireland Limited, Dublin 2 (IE)

(21) Appl. No.: 19/573,936

(22) Filed: Mar. 20, 2026

Related U.S. Application Data
(63) Continuation of application No. 17/801,389, filed on Feb. 24, 2021.

(30) Foreign Application Priority Data
Feb. 24, 2020 (EP) 20159161.7

(51) Int. Cl. A61K 31/4045; A61K 9/12

(52) U.S. Cl. CPC A61K 31/4045; A61K 9/12

(57) ABSTRACT

Aerosols of 5-methoxy-N, N-dimethyltryptamine (5-MeO-DMT) or a pharmaceutically acceptable salt thereof are provided which are useful for administration to a patient through an inhalation route. The aerosols have aerosol particle mass densities in the range of about 0.5 mg/l to about 12.5 mg/l.

### CROSS-REFERENCE TO RELATED APPLICATIONS

This application is a continuation of U.S. patent application Ser. No. 17/801,389, filed Aug. 22, 2022 which is a 371 National Phase Application of International Application No. PCT/EP2021/054502, filed Feb. 24, 2021, which claims benefit and foreign priority to European Application No. 20159161.7, filed Feb. 24, 2020. The contents of each are incorporated herein by reference in their entirety and for all purposes.

### TECHNICAL FIELD

The present invention relates to drug aerosols. More in particular, the invention relates to aerosols of 5-methoxy-N,N-dimethyltryptamine (5-MeO-DMT) or a pharmaceutically acceptable salt thereof which are useful for administration to a patient through an inhalation route, whereby the 5-MeO-DMT or a pharmaceutically acceptable salt thereof is delivered to the patient systemically via the lungs.

### BACKGROUND OF THE INVENTION

5-MeO-DMT is a naturally occurring serotonergic tryptamine which acts as a 5-HT1A and 5-HT2A receptor agonist. 5-MeO-DMT and compositions comprising 5-MeO-DMT besides other active components have a long history of recreational use, where their ability to induce intensely altered states of consciousness (including euphoria, trance, transcendence of time and space, spiritual experiences, dissolution of self-boundaries, or even near-death experiences; so called “psychedelic” effects) has been applied in spiritual or self-exploratory context.

The most commonly described route of administration for 5-MeO-DMT in the recreational context is inhalation into the lungs of “vapors” comprising 5-MeO-DMT ultimately leading to the absorption of 5-MeO-DMT into the bloodstream and systemic distribution. The “vapors” comprising 5-MeO-DMT are most commonly generated by exposing 5-MeO-DMT-containing materials to high temperatures over a longer period of time, e.g., in glass pipes using a torch lighter.

Based on its pharmacological activities, there has recently been an interest in potential medical uses of 5-MeO-DMT, for instance, investigating potential medical uses in human clinical trials. For such uses in human clinical trials, and for potential use in an approved medical product for treatment of patients, administration of 5-MeO-DMT in high purity is required.

The above described “vaporization” would be unsuitable for any medical application. It does not allow for the administration of defined amounts of 5-MeO-DMT. In many instances, even the exact 5-MeO-DMT content of the material subjected to “vaporization” and its purity are unknown.

The proportion of 5-MeO-DMT which is “vaporized” is likewise unknown, and the properties of the “vapors” are ill-defined.

Further, as indicated above, the conditions currently applied in the recreational context involve the exposure of 5-MeO-DMT to undefined high temperatures over a longer period of time. This induces the formation of thermal 5-MeO-DMT degradation products, which are also inhaled. Such degradation products have unknown pharmacological effects and they are potentially noxious. They also cause a harsh taste. A further disadvantage of the conditions currently applied in the recreational context to generate the 5-MeO-DMT “vapors” is that inhalation of those “vapors” can often lead to coughing, which prevents from intake of the total 5-MeO-DMT target dosage in a single inhalation and which limits the exposure duration of the lung tissues with 5-MeO-DMT and therefore its absorption.

Each of those issues independently and in combination contributes to inefficient and unpredictable systemic delivery of 5-MeO-DMT, which is not acceptable in the context of potential use of 5-MeO-DMT as a medication, as it can lead to suboptimal clinical efficacy and increased risk for side effects. For potential medical uses, e.g., for use in human clinical trials or for use in an approved medical product for treatment of patients, it is important to provide the complete or almost complete target dosage of 5-MeO-DMT to the patient in a single inhalation (i.e., within one deep breath), because the onset of psychedelic effects is so rapid that the patient will often not be able to accurately perform a second inhalation (i.e., take a second deep breath). The 5-MeO-DMT must be provided under well-controlled, standardized, and reproducible conditions. This has not been addressed in the prior art.

While thermally-generated condensation aerosols of some drugs and devices for the delivery of such aerosols have been disclosed, for instance, in U.S. Pat. No. 7,090,830 B2, EP1389098B1, and U.S. Pat. No. 8,955,512 B2, these patents do not teach or suggest aerosols containing 5-MeO-DMT. For the compounds tested, highly variable results are reported for the amount of degradation products (e.g., degradation product amounts of more than 80% to less than 1%), for the yield (e.g., yield from less than 25% to more than 90%) and for the physical properties (such as the mass median aerodynamic diameter) of the generated aerosols.

Against this background, there remains a need for a reproducible method for administration of 5-MeO-DMT or a pharmaceutically acceptable salt thereof, in particular, through an inhalation route. There is in particular a need for an aerosol of 5-MeO-DMT or a pharmaceutically acceptable salt thereof having a suitable aerosol particle mass density so that a therapeutically effective dose of the aerosol can be administered to a patient via a single inhalation.

### SUMMARY OF THE INVENTION

The present invention relates to an aerosol comprising (a) a pharmaceutically acceptable gas; (b) aerosol particles of 5-methoxy-N,N-dimethyltryptamine (5-MeO-DMT) or a pharmaceutically acceptable salt thereof, wherein the aerosol has an aerosol particle mass density of about 0.5 mg/l to about 12.5 mg/l, preferably of about 1.3 mg/l to about 10 mg/l, in particular of about 2 mg/l to about 9 mg/l. The pharmaceutically acceptable gas is preferably air.

The aerosol particles preferably contain less than 1 wt % impurities, in particular less than 0.5 wt % impurities. They furthermore preferably contain less than 0.5 wt % 5-MeO-DMT degradation products, in particular less than 0.2 wt % 5-MeO-DMT degradation products resulting from a chemical modification of 5-MeO-DMT as a result of a chemical reaction during aerosol formation.

In a further preferred aspect of the invention, the aerosol essentially consists of (a) air; (b) aerosol particles of 5-MeO-DMT or a pharmaceutically acceptable salt thereof.

The aerosol particles preferably contain 5-MeO-DMT in the form of the free base.

The aerosol is preferably characterized by a mass median aerodynamic diameter of less than 3 micron and more than 0.1 micron, in particular by a mass median aerodynamic diameter of less than 2 micron and more than 0.1 micron.

The aerosol may be formed by a) exposing a thin layer of 5-MeO-DMT or a pharmaceutically acceptable salt thereof, configured on a solid support, to thermal energy, and b) passing air over the thin layer of 5-MeO-DMT to produce aerosol particles. The thin layer may have a thickness of less than about 10 μm, in particular less than about 7.5 μm. It may have a thickness in the range of about 0.1 μm to about 10 μm, in particular in the range of about 0.3 μm to about 7.5 μm.

The thin layer of 5-MeO-DMT, configured on a solid support, may be exposed to thermal energy via the air passing over the thin layer. Alternatively, the thin layer of 5-MeO-DMT, configured on a solid support, may be exposed to thermal energy via the solid support.

The air passing over the thin layer may have a temperature in the range of about 180° C. to about 260° C. The air passing over the thin layer may in particular have a temperature of about 210° C. and pass over the thin layer at a rate of about 12 l/min for a duration of about 15 seconds.

The aerosol particles may be contained in a volume of equal or less than about 3 liters, such as a volume of about 1.5 to about 2 liters, in particular in a volume of about 2 to about 3 liters. The aerosol is in particular for use in therapy. It is preferably delivered to a patient via a single inhalation.

### DESCRIPTION OF THE FIGURES

FIG. 1 is a diagram obtained by plotting the peak area, determined by HPLC, vs the concentration of 5-MeO-DMT in a sample.

FIG. 2 shows a Next Generation Impactor (NGI) USP <601> Apparatus 6.

FIG. 3 shows a typical dose sampling apparatus USP <601> Apparatus A.

### DETAILED DESCRIPTION OF THE INVENTION

The present invention aims at providing 5-MeO-DMT or a pharmaceutically acceptable salt thereof in a form suitable for inhalation in a medical context. The invention in particular provides 5-MeO-DMT and pharmaceutically acceptable salts thereof in the form of aerosols. These aerosols have a suitable aerosol particle mass density so that a therapeutically effective dose of the aerosol can be administered to a patient via a single inhalation.

Aerosols useful in the present invention can be formed using thermal energy. When using thermal energy to form an aerosol of a compound, it is very difficult to predict which conditions are suitable for safe, efficient and predictable aerosolization, in particular if the aerosol is to be used for systemic delivery of that compound to a patient via the lungs. Relevant variables in this context include a) the dose of the compound, b) the morphological state in which that compound is made available for aerosolization (e.g. in crystal form, or in form as a thin layer), c) the amount of thermal energy to which the compound is exposed (defined by temperature and duration of exposure), and d) the volume of air introduced to create the aerosol (defined by flow rate and duration of air flow).

In a general sense, the present invention aims at providing compositions and methods for safe, efficient and predictable systemic delivery of 5-MeO-DMT or a pharmaceutically acceptable salt thereof to a patient through inhalation. In the context of the technical problem, “safe” means that the aerosol particles should contain only a very small amount of impurities and 5-MeO-DMT degradation products, “efficient” means that the dosage is aerosolized to a defined extent and preferably almost completely or completely, that the aerosol has desirable physical properties for delivery of the 5-MeO-DMT or a pharmaceutically acceptable salt thereof systemically via the lungs mainly via absorption in the pulmonary alveoli, and that the aerosol can be inhaled by the patient in a single inhalation (i.e., within one deep breath), and “predictable” means that there should be almost no or no variability in the amount of degradation products, in the extent of aerosolization, and in the physical properties of the aerosol.

In a more specific sense, the present invention aims at providing specific parameters for a) the dosage amount of the 5-MeO-DMT, b) the morphological state in which 5-MeO-DMT is made available for aerosolization, b) the amount of thermal energy to which 5-MeO-DMT is exposed, and c) the volume of air introduced to create the 5-MeO-DMT aerosol.

The inventor has recognized that a safe, efficient and predictable systemic delivery of 5-MeO-DMT to a patient can be achieved by providing 5-MeO-DMT in the form of an aerosol. The aerosol of the invention contains a defined mass of 5-MeO-DMT per unit volume, with a defined mass median aerodynamic diameter, and a defined maximum amount of impurities, such as 5-MeO-DMT degradation products. The aerosol is administered through inhalation into the lungs, preferably in a single inhalation.

A suitable aerosol can be achieved by a) providing the therapeutically effective amounts of 5-MeO-DMT as a thin layer, on a solid support, b) exposing the thin 5-MeO-DMT layer to elevated controlled temperatures for a short duration of time, and c) providing a controlled amount of air so that an aerosol is formed.

The present invention provides compositions and methods to provide aerosols comprising 5-MeO-DMT that are useful in inhalation therapy of patients, whereby the therapeutically effective 5-MeO-DMT dosage amount contained in a composition is aerosolized completely or almost completely, the aerosol particles contain only a very small amount of impurities and 5-MeO-DMT degradation products, the aerosol has desirable physical properties for delivery of the 5-MeO-DMT systemically via the lungs mainly via absorption in the respiratory pulmonary alveoli, and the aerosol can be inhaled by the patient in a single inhalation, with limited variability in the extent of aerosolization, the amount of degradation products, and in the physical properties of the aerosol, all of which has not been achieved in the state of the art.

### Definitions

As used in the context of the present invention, unless otherwise noted, the term “5-MeO-DMT” refers to the free base 5-MeO-DMT. It is contemplated that pharmaceutically acceptable salts of 5-MeO-DMT may also be used. An example for such a salt is the hydrochloride. The appropriate weight amount of a salt to be administered can be calculated from the weight amount of the free base, assuming that equimolar amounts are used.

As used herein, “aerosol” means a stable system consisting of a gaseous medium (a pharmaceutically acceptable gas, such as air) and miniscule suspended solid and/or liquid particles, herein also referred to as droplets.

As used in the context of the present invention, unless otherwise noted, the term “degradation product” refers to a compound resulting from a chemical modification of 5-MeO-DMT as a result of a chemical reaction during aerosol formation. Such reaction includes, without limitation, oxidation.

When a percentage of a “degradation product” is described in the context of the present invention, then this refers to the quantity of 5-MeO-DMT degradation products present in a sample divided by the quantity of 5-MeO-DMT plus 5-MeO-DMT degradation products present in the sample multiplied by 100%, i.e., (Sum of quantities of all 5-MeO-DMT degradation products present in the sample)/((Quantity of 5-MeO-DMT present in the sample)+(Sum of quantities of all 5-MeO-DMT degradation products present in the sample))×100%.

As used herein, the term “impurity” refers to unwanted compounds contaminating a sample of 5-MeO-DMT (or of a pharmaceutically acceptable salt thereof). Impurities may be contained in the starting material before aerosol formation or may be degradation products.

As used in the context of the present invention, unless otherwise noted, the term “purity” refers to 100% minus the percent of all 5-MeO-DMT degradation products and all other impurities present, i.e., (100%−(Sum of quantities of all 5-MeO-DMT degradation products present+Sum of quantities of all other impurities present)/(Quantity of 5-MeO-DMT present+Sum of quantities of all 5-MeO-DMT degradation products present+Sum of quantities of all other impurities present)×100%.

As used in the context of the present invention, a “patient” to be treated is a human subject who has been diagnosed in accordance with accepted medical practice by a licensed professional (e.g., a physician) as suffering from a disease, disorder or condition, and who may seek or be in need of treatment, requires treatment, is receiving treatment, or will receive treatment.

As used in the context of the present invention, unless otherwise noted, the terms “treating” and “treatment” and “therapy” shall include the management and care of a patient for the purpose of combating a disease, condition, or disorder and includes the administration of compounds and methods according to the present invention to alleviate the signs and/or symptoms or eliminate the disease, condition, or disorder.

As used in the context of the present invention, unless otherwise noted, the term “therapeutically effective amount” shall mean the amount of active compound or pharmaceutical ingredient that elicits a clinical response in a patient, which includes alleviation of the signs and/or symptoms of the disease, condition or disorder being treated.

As used in the context of the present invention, unless otherwise noted, the term “administration” shall mean the introduction of an amount, which may be a predetermined amount, of active compound or pharmaceutical ingredient into a patient via inhalation into the lungs.

As used in the context of the present invention, unless otherwise noted, the terms “dose” and “dosage” and “dosage amount” shall mean the amount of active compound or pharmaceutical ingredient which is administered to a patient in an individual administration.

As used in the context of the present invention, unless otherwise noted, the term “mass median aerodynamic diameter” (MMAD), is the diameter at which 50% of the particles present in an aerosol are larger than this calculated diameter, and 50% are smaller.

As used in the context of the present invention, unless otherwise noted, the term “aerosol particle mass density” refers to the mass of aerosol particles per unit volume of aerosol.

As used in the context of the present invention, unless otherwise noted, the term “aerosol particle formation rate” refers to the aerosolized mass of 5-MeO-DMT per unit of aerosolization time.

Note that in this specification, when ranges are set forth, such as “about 1 mg to about 25 mg”, the inventor contemplates all discrete values within that range, some of which are specifically mentioned, but all of which are not-simply for the purpose of brevity.

In a composition aspect of the present invention, a composition for delivery of a therapeutically effective amount of 5-MeO-DMT comprises an aerosol, wherein the aerosol is formed by a) exposing a thin layer of 5-MeO-DMT, configured on a solid support, to thermal energy, and b) passing air over the thin layer of 5-MeO-DMT; wherein said aerosol has one or more of the following features: 1) it contains aerosol particles which are characterized by a mass median aerodynamic diameter of less than 3 micron, 2) it contains aerosol particles which are characterized by less than 1% wt impurities and less than 0.5% 5-MeO-DMT degradation products, 3) it can be delivered to a patient via a single inhalation.

In a method aspect of the present invention, a therapeutically effective amount of 5-MeO-DMT is delivered to a patient through an inhalation route. The method comprises: a) exposing a thin layer of 5-MeO-DMT, configured on a solid support, to thermal energy, and b) passing air over the thin layer of 5-MeO-DMT; wherein said aerosol has one or more of the following features: 1) it contains aerosol particles which are characterized by a mass median aerodynamic diameter of less than 3 micron, 2) it contains aerosol particles which are characterized by less than 1% wt impurities and less than 0.5% wt 5-MeO-DMT degradation products, 3) it can be delivered to a patient via a single inhalation.

In the composition, method and kit aspects of the present invention, the generation of aerosol particles characterized by a mass median aerodynamic diameter of less than 3 microns, with less than 1% wt impurities and less than 0.5% wt 5-MeO-DMT drug degradation products, in an aerosol volume which can be delivered to a patient via a single inhalation, is achieved by defining a) the dosage amount of 5-MeO-DMT contained in the thin layer of 5-MeO-DMT, b) the thickness of the thin layer of the 5-MeO-DMT, c) the thermal energy to which the thin layer of 5-MeO-DMT is exposed (defined by temperature and duration of exposure), and d) the total amount of the air which passes over the thin layer of 5-MeO-DMT (defined by airflow rate and duration of airflow).

Preferably, in the composition, method and kit aspects of the present invention, the thin layer of 5-MeO-DMT is exposed to thermal energy via the air passing over the thin layer, in which case that air is heated. The heated air passing over the thin layer may have a temperature in the range of about 180° C. to about 260° C. The air passing over the thin layer may in particular have a temperature of about 210° C.

Alternatively, in the composition, method and kit aspects of the present invention, the thin layer of 5-MeO-DMT is exposed to thermal energy via the solid support, in which case the air passing over the thin layer is not heated, but the solid support is heated. The heated solid support may have a temperature in the range of about 180° C. to about 420° C.

Preferably, in the composition, method and kit aspects of the present invention, the 5-MeO-DMT used for formation of the thin layer, on the solid support, is highly pure, with a purity of at least 99%, preferably at least 99.5%.

Preferably, in the composition, method and kit aspects of the present invention, the dosage amount of 5-MeO-DMT contained in the thin layer of 5-MeO-DMT, configured on the solid support, is from about 1 mg to about 25 mg, preferably from about 2 mg to about 20 mg, more preferably from about 4 mg to about 20 mg. Useful specific amounts are, e.g., about 4 mg, about 6 mg, about 8 mg, about 10 mg, about 12 mg, about 14 mg, about 16 mg, about 18 mg, and about 20 mg. Preferred specific amounts are e.g. about 6 mg, about 12 mg, and about 18 mg.

Solid supports, on which 5-MeO-DMT or a pharmaceutically acceptable salt thereof is provided, can have a variety of shapes. Examples of such shapes include, without limitation, cylinders of less than 1.0 mm in diameter, boxes of less than 1.0 mm thickness and virtually any shape permeated by small (e.g., less than 1.0 mm-sized) pores. Preferably, solid supports provide a large surface to volume ratio (e.g., greater than 100 per meter) and a large surface to mass ratio (e.g., greater than 1 cm² per gram).

A solid support of one shape can also be transformed into another shape with different properties. For example, a flat sheet of 0.25 mm thickness has a surface to volume ratio of approximately 8,000 per meter. Rolling the sheet into a hollow cylinder of 1 cm diameter produces a support that retains the high surface to mass ratio of the original sheet but has a lower surface to volume ratio (about 400 per meter).

A number of different materials are used to construct the solid supports. Classes of such materials include, without limitation, metals, inorganic materials, carbonaceous materials and polymers. The following are examples of the material classes: aluminum, silver, gold, stainless steel, copper and tungsten; silica, glass, silicon and alumina; graphite, porous carbons, carbon yarns and carbon felts; polytetrafluoroethylene and polyethylene glycol. Combinations of materials and coated variants of materials are used as well.

Where aluminum is used as a solid support, aluminum foil is a suitable material. Examples of silica, alumina and silicon based materials include amphorous silica S-5631 (Sigma, St. Louis, Mo.), BCR171 (an alumina of defined surface area greater than 2 m2/g from Aldrich, St. Louis, Mo.) and a silicon wafer as used in the semiconductor industry. Carbon yams and felts are available from American Kynol, Inc., New York, N.Y.

Preferably, the thickness of the thin layer of the 5-MeO-DMT, configured on the solid support, is less than about 10 μm, in particular less than about 7.5 μm. It may have a thickness in the range of about 0.1 μm to about 10 μm, in particular in the range of 0.3 μm to 7.5 μm.

Preferably, in the composition, method and kit aspects of the present invention, the total amount of the air passing over the thin layer of 5-MeO-DMT is defined by a flow rate of between about 6 liters per minute and about 80 liters per minute, such as about 6 liters per minute and about 40 liters per minute, preferable between about 8 liters per minute and about 16 liters per minute and the duration of airflow is chosen so that the total volume of aerosol does not exceed about 3 liters, preferably is between about 2 liters and 3 liters. E.g., at an airflow rate of about 6 liters per minute, the duration of airflow should be less than about 30 seconds. A useful specific airflow rate and duration is about 12 liters per minute and about 15 seconds, leading to an aerosol volume of about 3 liters. Another useful specific airflow rate and duration is 10 liters per minute and about 15 seconds, leading to leading to an aerosol volume of about 2.5 liters. Another useful specific airflow rate and duration is 8 liters per minute and about 15 seconds, leading to leading to an aerosol volume of about 2 liters. Another useful specific airflow rate and duration is 10 liters per minute and about 12 seconds, leading to leading to an aerosol volume of about 2 liters.

The aerosol formation rate is greater than 0.1 mg/sec.

In the composition, method and kit aspects of the present invention, the aerosol has an aerosol particle mass density of about 0.5 mg/l to about 12.5 mg/l, preferably of about 1.3 mg/l to about 10 mg/l, in particular of about 2 mg/l to about 9 mg/l.

In the composition, method and kit aspects of the present invention the 5-MeO-DMT aerosol particles are characterized by a mass median aerodynamic diameter of less than 3 micron and more than 0.1 micron, preferably of less than 2.5 micron and more than 0.1 micron, most preferably of less than 2 micron and more than 0.1 micron.

In the composition, method and kit aspects of the present invention the 5-MeO-DMT aerosol particles are characterized by less than 1% wt impurities, preferably by less than 0.5% wt impurities. In the composition, method and kit aspects of the present invention the 5-MeO-DMT aerosol particles are characterized by less than 0.5% wt 5-MeO-DMT degradation products, preferably by less than 0.2% wt 5-MeO-DMT degradation products.

In a specific composition aspect of the present invention a composition for delivery of a therapeutically effective amount of 5-MeO-DMT comprises an aerosol, wherein the aerosol is formed by a) exposing a dosage amount of 12 mg 5-MeO-DMT, configured as a thin layer of less than 5 micron thickness on a solid support, to a temperature of 210° C. via passing heated air over the thin layer for a duration of 15 seconds; wherein said aerosol has one or more of the following features: 1) it contains aerosol particles which are characterized by a mass median aerodynamic diameter of less than 3 micron, 2) it contains aerosol particles which are characterized by less than 1% impurities and less than 0.5% wt 5-MeO-DMT degradation products, 3) it can be delivered to a patient via a single inhalation.

A skilled person, knowing the aerosol characteristics and the aerosolization conditions defined in the present invention, can identify suitable vaporization devices or systems, which lead to the required aerosol characteristics. Examples of such suitable vaporization devices or systems include e.g. the Volcano Medic Vaporization System with the associated dosing capsules with drip pad (Storz & Bickel, Germany; as disclosed in e.g. EP 0 933 093 B1, and EP 1 884 254 B1 and Registered Community Design 003387299-0001) and the Staccato device (Alexza Pharmaceuticals, Mountain View, USA; as disclosed e.g. in U.S. Pat. No. 7,458,374 B2, U.S. Pat. No. 9,370,629 B2 and U.S. Pat. No. 9,687,487 B2).

The aerosol generated may be collected in a balloon and inhaled by the patient from the balloon.

### EXAMPLES

### Example 1. 5-MeO-DMT Aerosol Generation and Administration

### Volcano Medic Vaporization System

A 5-MeO-DMT aerosol was generated by volatilization of the drug by way of the Volcano Medic Vaporization System (Storz & Bickel, Germany). The device consists of a hot air generator and a detachable valve balloon from which the aerosol is inhaled by the patient. The hot air generator can generate temperatures adjustable between about 40° C. to about 210° C., with an airflow rate of about 12 liters per minute. The central part of the device is the dosing capsule to which relevant doses of 5-MeO-DMT in an ethanol solution are applied and which is then applied into the filling chamber of the device, where it is heated via the hot air. The dosing capsules contain a small disc made of tightly packed stainless-steel wire mesh (called the drip pad or liquid pad). The bottom and the lid of the dosing capsules have holes, allowing airflow through the dosing capsules. The dosing capsules and drip pad have the following characteristics, based on measurements of 10 sample capsules:

### Example 1, Table 1. Characteristics of Dosing Capsules and Drip Pads

| Item | Mean (standard deviation)¹ | |
|---|---|---|
| Dosing capsule without lid (outer diameter) | 14.3 mm | (0.03 mm) |
| Dosing capsule without lid (height) | 8.0 mm | (0.03 mm) |
| Dosing capsule without lid (weight) | 236.3 mg | (2.6 mg) |
| Dosing capsule with lid (weight) | 361.9 mg | (2.6 mg) |
| Dosing capsule with lid and with drip | 1323.4 mg | (52.5 mg) |
| pad (weight) | | |
| Lid (outer diameter) | 14.4 mm | (0.06 mm) |
| Lid (height) | 3.2 mm | (0.03 mm) |
| Lid (weight) | 125.6 mg | (0.8 mg) |
| Number of holes (lid) | 33 | (0) |
| Number of holes (dosing capsule base) | 33 | (0) |
| Diameter of holes in lid and base | 1138 μm | (57 μm) |
| Drip pad (weight) | 961.9 mg | (52.2 mg) |
| Stainless steel wire in drip pad (diameter) | 113 μm | (12 μm) |
| Stainless steel wire in drip pad (length) | 1062.0 cm | (55.8 cm) |
| Stainless steel wire in drip pad | 37.78 cm² | (1.99 cm²) |
| (calculated surface area) | | |
| Drip pad weight/length index (mg/cm) | 0.906 | (0.013) |

All measurements show the mean and standard deviation for measurements of 10 capsules, except for the diameter of holes in lid and base, which is based on 40 measurements across 2 capsules and for diameter of the stainless steel wire in drip pad, which is based on 40 measurements in different locations on the stainless steel wire.

### 5-MeO-DMT Aerosol Generation and Administration

Step 1: A stock solution of 5-MeO-DMT free base in 100% ethanol is prepared in a volumetric flask, so that the target dosage of 5-MeO-DMT free base to be administered via inhalation to the patient is contained in a solution volume of 200 μl. Typical target dosages are from 1 mg to 25 mg 5-MeO-DMT. E.g. for a target dosage of 18 mg 5-MeO-DMT, 90 mg of 5-MeO-DMT will be dissolved in 100% ethanol for a final solution volume of 1 ml. Aliquots of the stock solution can then be stored in vials until further use.
Step 2: 200 μl of the solution is transferred to a dosing capsule containing the drip pad (Storz & Bickel, Germany), and then the dosing capsule is closed with its lid.
Step 3: The dosing capsule filled with the 5-MeO-DMT ethanol solution is transferred to the filling chamber of a first Volcano Medic Vaporizer, which has been pre-heated with the temperature set at 55° C. Then the airflow of the vaporizer is switched on for 60 seconds at the pre-set rate of about 12 l/min. The heated air will flow through the dosing capsule, allowing the ethanol to evaporate, with the target dosage of 5-MeO DMT being left in the capsule, as a thin layer covering the stainless-steel wire mesh. Accurate preparation of the dosing capsule can be confirmed by demonstrating that the final weight increase of the capsule compared to the weight of the empty capsule corresponds to the target dosage of 5-MeO-DMT.
Step 4: The prepared dosing capsule is removed from the filling chamber. It is then transferred to the filling chamber of a second Volcano Medic Vaporizer, which has been pre-heated with the temperature set at 210° C. and the airflow on for at least 5 minutes and then turned off immediately prior to transfer. An inhalation balloon with a valve (Storz & Bickel, Germany) is mounted on the socket of the filling chamber, the filling chamber is closed tightly and immediately afterwards the airflow is switched on for exactly 15 seconds at the pre-set flow rate of about 12 l/min, and then turned off. This will allow the full dose of 5-MeO-DMT to aerosolize and be distributed in approximately 3 liters of air in the inhalation balloon. Accurate aerosolization of the 5-MeO-DMT can be confirmed by demonstrating that the capsule weight has returned to about its initial weight.
Step 5: The balloon is then disconnected from the filling chamber, which automatically closes the valve. After attachment of the mouthpiece to the balloon, the aerosol is ready for immediate administration to the patient, or for immediate analytical procedures.
Step 6: To prepare for the administration, the patient is asked to initially perform 1-2 deep inhalations with full exhalations, ending this sequence with a deep exhalation. Then, with the mouthpiece firmly held against the lips, the full and complete volume of the inhalation balloon is inhaled in one inhalation, holding the breath for 10 (±2.5) seconds, followed by a normal exhalation. After completing the inhalation procedure, the patient will be instructed to lie down.

### Example 2. Loading of Dosing Capsules with 5-MeO-DMT, and Determination of Aerosolized Dose

Triplicates of dosing capsules with a 5-MeO-DMT target dosage of 2 mg and 18 mg were prepared as described in Example 1, Steps 1 to 3, using a 5-MeO-DMT stock solution stored as 200 μl aliquots in single use vials. For confirmation of accurate loading of the dosing capsules with the target dosage of 5-MeO-DMT, the baseline weight of the empty capsules was subtracted from the weight of the capsules after Step 3, confirming that about 94% of the target dose of 5-MeO-DMT was loaded on the capsules, with only minimal variability (Example 2, Table 1). The fact that not 100% of the target dose was achieved can be explained by loss of material in the vials used for storage of the 5-MeO-DMT stock solution (which had about 2 μl residual volume) and by additional loss in the pipette tips used for transfer of the solution from the vials to the capsules. Such loss however can be prevented by pipetting from a larger volume of stock solution and by optimizing pipetting technique.

5-MeO-DMT was then aerosolized from the dosing capsules as described in Example 1, Steps 4 and 5. For confirmation of accurate aerosolization of 5-MeO-DMT from the dosing capsules, the weight after Step 4 was subtracted from the weight after Step 3, confirming that between 96% and 100% of the loaded dose was aerosolized (Example 2, Table 1).

### Example 2, Table 1. Loading of Dosing Capsules with 5-MeO-DMT and Subsequent Aerosolization

| | 2 mg—1 | 2 mg—2 | 2 mg—3 | 18 mg—1 | 18 mg—2 | 18 mg—3 |
|---|---|---|---|---|---|---|
| Empty (mg) | 1291.1 | 1312.1 | 1255.5 | 1288.5 | 1225.9 | 1297.4 |
| After Step 3 (mg) | 1292.9 | 1314.0 | 1257.4 | 1305.4 | 1242.6 | 1314.4 |
| Loaded dose¹ (mg) | 1.8 | 1.9 | 1.9 | 17.0 | 16.7 | 17.0 |
| % of target dose | 92.0 | 94.0 | 93.0 | 94.3 | 92.9 | 94.3 |
| After Step 4 | 1291.1 | 1312.1 | 1255.5 | 1289.1 | 1226.5 | 1298.1 |
| Aerosolized dose² (mg) | 1.9 | 1.9 | 1.9 | 16.3 | 16.2 | 16.3 |
| % of loaded dose | 100.5 | 99.5 | 99.5 | 96.3 | 96.5 | 95.9 |

Weights are shown in mg for triplicates of dosing capsules with a 5-MeO—DMT target dosage of 2 mg and 18 mg.
¹Loaded dose = Empty weight—Weight after Step 3.
²Aerosolized dose = Weight after Step—3Weight after Step 4.

Instead of determining the loading of dosing capsules with the 5-MeO-DMT target dose by weighing the dosing capsules before and after formation of the 5-MeO-DMT layer, alternatively the loading can also be determined by extracting the drug from the dosing capsule and measuring the amount analytically.

Instead of determining the extent of aerosolization of the 5-MeO-DMT target dose from the capsules by weighing the capsules before and after formation of the 5-MeO-DMT layer and again after aerosolization, alternatively the emitted dose of 5-MeO-DMT can be determined by delivering the 5-MeO-DMT-containing aerosol into a confined chamber and measuring the amount of 5-MeO-DMT collected in the chamber analytically.

### Example 3. Thickness of 5-MeO-DMT Layer

The thickness of the 5-MeO-DMT layer covering the stainless-steel wire mesh after evaporation of the ethanol solvent can be calculated as follows: 5-MeO-DMT layer thickness (μm)=5-MeO-DMT loaded dose (mg)/[5-MeO-DMT density (mg/cm³)×wire surface area (cm²)]*10000. The wire surface area can be calculated based on the length of the wire (which can be measured, or calculated, from the weight of the wire mesh) and diameter of the wire (which can be measured).

For the dosing capsules as prepared in Example 2, the following layer thickness was determined:

### Example 3, Table 1. Thickness of 5-MeO-DMT Layer for Dosing Capsules as Prepared in Example 2

| | 2 mg—1 | 2 mg—2 | 2 mg—3 | 18 mg—1 | 18 mg—2 | 18 mg—3 |
|---|---|---|---|---|---|---|
| Loaded dose (mg)¹ | 1.8 | 1.9 | 1.9 | 17.0 | 16.7 | 17.0 |
| Wire surface are (cm²) | 36.58 | 37.35 | 35.29 | 36.49 | 34.21 | 36.81 |
| Thickness (μm) | 0.46 | 0.46 | 0.48 | 4.23 | 4.45 | 4.19 |

¹Loaded dose from Example 2; For the calculations a 5-MeO—DMT density of 1100 mg/cm³ was assumed.

For a target loaded dose of 2 mg, the thickness of the 5-MeO-DMT layer based on an average wire surface area of 37.78 cm² can be calculated as 0.48 μm; and for a target loaded dose of 20 mg, the thickness of the 5-MeO-DMT layer can be calculated as 4.8 μm.

### Example 4. Determination of Aerosol Particle Formation Rate and Aerosol 5-MeO-DMT Mass Density

The aerosol particle formation rate can be calculated as follows: Aerosol particle formation rate=Aerosolized dose/Aerosolization time. For the aerosolized dose data and the aerosolization time of 15 seconds from Example 2, the following aerosol particle formation rate was determined:

### Example 4, Table 1. Aerosol Particle Formation Rate for Dosing Capsules as Prepared in Example 2

| | 2 mg—1 | 2 mg—2 | 2 mg—3 | 18 mg—1 | 18 mg—2 | 18 mg—3 |
|---|---|---|---|---|---|---|
| Aerosolized dose (mg)¹ | 1.9 | 1.9 | 1.9 | 16.3 | 16.2 | 16.3 |
| Aerosolparticle | 0.12 | 0.12 | 0.12 | 1.09 | 1.08 | 1.09 |
| formation rate (mg/s) | | | | | | |

¹Aerosolized dose from Example 2

For a target aerosolized dose of 2 mg and an aerosolization time of 15 seconds, the aerosol particle formation rate can be calculated as 0.13 mg/s; and for a target aerosolized dose of 20 mg and an aerosolization time of 15 seconds, the particle formation rate can be calculated as 1.33 mg/s.

The aerosol 5-MeO-DMT mass density can be calculated as follows: Aerosol 5-MeO-DMT mass density =Aerosolized dose/Aerosol volume. For the aerosolized dose data and the aerosol volume of about 3 liters from Example 2, the following aerosol 5-MeO-DMT mass density was determined:

### Example 4, Table 2. Aerosol 5-MeO-DMT Mass Density for Dosing Capsules as Prepared in Example 2

| Weights/Dose | 2 mg—1 | 2 mg—2 | 2 mg—3 | 18 mg—1 | 18 mg—2 | 18 mg—3 |
|---|---|---|---|---|---|---|
| Aerosolized dose (mg)¹ | 1.9 | 1.9 | 1.9 | 16.3 | 16.2 | 16.3 |
| Aerosol 5-MeO—DMT | 0.62 | 0.62 | 0.62 | 5.45 | 5.38 | 5.43 |
| mass density (mg/l) | | | | | | |

¹Aerosolized dose from Example 2.

For a target aerosolized dose of 2 mg in 3 liters, the aerosol 5-MeO-DMT mass density can be calculated as 0.66 mg/l; for a target aerosolized dose of 20 mg in 3 liters, the aerosol 5-MeO-DMT mass density can be calculated as 6.66 mg/l. For a target aerosolized dose of 2 mg in 2 liters, the aerosol 5-MeO-DMT mass density can be calculated as 1 mg/l; for a target aerosolized dose of 20 mg in 2 liters, the aerosol 5-MeO-DMT mass density can be calculated as 10 mg/l.

### Example 5. HPLC Assay for Determination of Purity of 5-MeO-DMT

An HPLC assay was developed to allow determination of the purity of 5-MeO-DMT. The assay was tested for linearity and precision. Based on the results, the method was considered as fit for purpose.

The Following Method Parameters were Used:
Instrument: A suitable HPLC system equipped with UV detection, linked to the laboratory data handling system
Column: ACE C18 (150×4.6×3 μm)
Injection Volume: 5 μl
Flow Rate: 0.75 ml/minute
Detector: UV at 227 nm
Run Time: 25 minutes
Column Temperature: 30° C.
Diluent: Methanol
Mobile Phase A: 0.013M Ammonium acetate in water
Mobile Phase B: Acetonitrile

### Example 5, Table 1. Gradient

| Time (minutes) | % Mobile Phase A | % Mobile Phase B |
|---|---|---|
| 0.0 | 80 | 20 |
| 18.0 | 26 | 74 |
| 20.0 | 26 | 74 |
| 20.1 | 80 | 20 |
| 25.0 | 80 | 20 |

Typical retention time of 5-MeO-DMT: 5.5 min

### Testing of the HPLC Method for Linearity:

A stock solution of 5-MeO-DMT was prepared in methanol. A nominal concentration of 0.15 mg/ml was taken.

### Example 5, Table 2. Testing of the HPLC Method for Linearity

| | Actual | | | | |
|---|---|---|---|---|---|
| | concentration | Peak Area | | | |
| % Nominal | (mg/ml) | Injection 1 | Injection 2 | mean | % RD |
| 150 | 0.226 | 132.511 | 134.435 | 133.473 | 1.4 |
| 125 | 0.181 | 109.305 | 108.094 | 108.700 | −1.1 |
| 100 | 0.151 | 91.466 | 92.675 | 92.070 | 1.3 |
| 80 | 0.121 | 73.543 | 72.295 | 72.919 | −1.7 |
| 50 | 0.075 | 46.871 | 46.891 | 46.881 | 0.0 |
| 25 | 0.038 | 23.965 | 24.056 | 24.011 | 0.4 |
| 10 | 0.015 | 9.675 | 9.706 | 9.690 | 0.3 |
| 5 | 0.008 | 4.670 | 4.694 | 4.682 | 0.5 |
| 1 | 0.000 | 0.982 | 0.989 | 0.985 | 0.7 |
| 0.1 | 0.002 | 0.468 | 0.472 | 0.470 | 0.8 |
| 0.01 | 0.000 | 0.097 | 0.095 | 0.096 | −2.1 |

All duplicate injections were within ±2%

### Linearity of the HPLC-Method

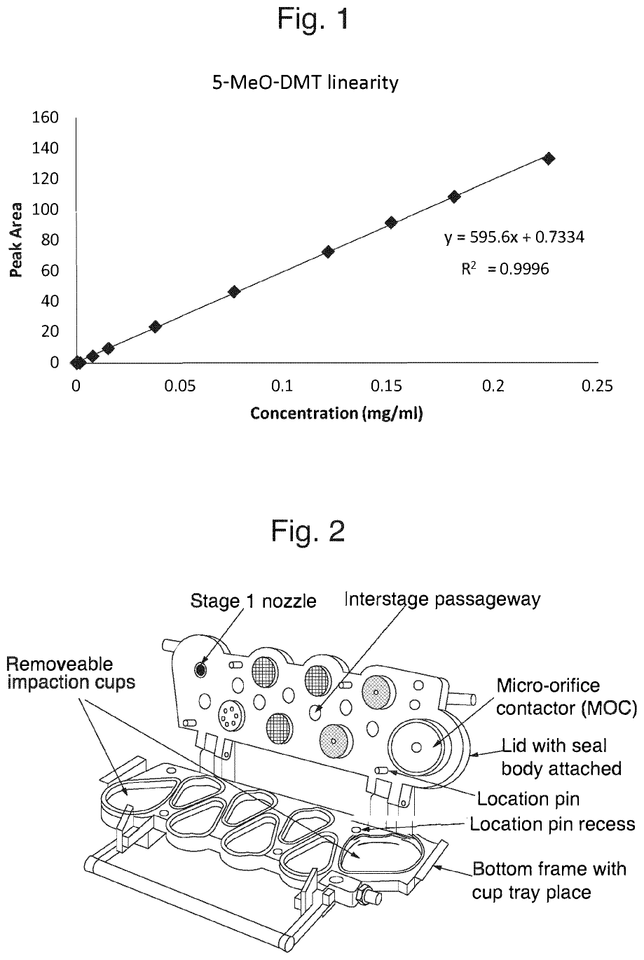

Y intercept % at nominal concentration was determined to be 0.8%. Method is deemed linear, as shown in FIG. 1.

### Testing of the HPLC Method for Precision:

Six sample solutions were prepared at nominal concentration (12-18 mg in 100 ml methanol). The purity results were as follows:

### Example 5, Table 3. Testing of the HPLC Method for Precision

| Precision | Purity (% area) |
|---|---|
| 1 | 99.21 |
| 2 | 99.02 |
| 3 | 99.18 |
| 4 | 99.21 |
| 5 | 99.17 |
| 6 | 99.17 |
| Average | 99.16 |
| SD | 0.07 |
| RSD (%) | 0.07 |

Acceptance criteria for purity values across the six samples would be 1% RSD, the actual reading was 0.07%. Therefore, the analytical method is considered to exhibit adequate precision.

### Example 6. Evaluation of Purity and Degradation Products of 5-MeO-DMT Aerosol

Duplicates of dosing capsules with a 5-MeO-DMT target dosage of 18 mg were prepared as described in Example 1, Steps 1 to 3, using a stock solution of 180.7 mg 5-MeO-DMT free base in 2 ml of ethanol (90.4 mg/ml), of which 200 μl were pipetted onto the drip pad in the capsules. The purity of the 5-MeO-DMT starting material, as determined by HPLC, was 99.605%, with three minor fractions of impurities (Example 6, Table 1).

### Example 6, Table 1. Purity of 5-MeO-DMT Starting Material

| | Retention Time | Area | Relative Area |
|---|---|---|---|
| Peak Name | (min) | (mAU*min) | (%) |
| 5-MeO-DMT | 6.144 | 125.808 | 99.605 |
| Impurity 1 | 7.659 | 0.125 | 0.099 |
| Impurity 2 | 14.128 | 0.019 | 0.015 |
| Impurity 3 | 14.337 | 0.354 | 0.281 |

5-MeO-DMT was then aerosolized from the dosing capsules as described in Example 1, Steps 4 and 5, except that only one Volcano Medic Vaporizer was used (i.e., the vaporizer in step 3 and step 4 was the same, with pre-heating between the capsule preparation and the aerosol generation performed according to the instructions).

For purity analysis of the aerosol, each replicate valve balloon containing the aerosol was connected to a Solid Phase Extraction (SPE) cartridge (Discovery® DSC-18). A vacuum was then applied until the balloon was fully deflated. 4 aliquots of 5 ml methanol were added to the cartridge and the extracts were analysed neat by HPLC. Extract 1 was further diluted (1 ml to 10 ml) to achieve a response in the linear range.

For Replicate 1, Extract 1 (Example 6, Table 1), it was found that the purity of the aerosol was even higher than the purity of the starting material (99.710% vs. 99.605%), that the pre-existing Impurities 2 and 3 were undetectable while pre-existing Impurity 1 only minimally increased (0.206% vs. 0.099%), and that only a minimal amount of new 5-MeO-DMT degradation products occurred (Degradation product 1:0.039%, Degradation product 2:0.044%), with a total percentage of 5-MeO-DMT degradation products in the aerosol of 0.19% (including the additional amount of Impurity 1). The results for the other replicate were very similar and the results for the other extracts did not change the conclusions.

### Example 6, Table 2. Purity of 5-MeO-DMT Aerosol, Replicate 1, Extract 1

| | Retention Time | Area | Relative Area |
|---|---|---|---|
| Peak Name | (min) | (mAU*min) | (%) |
| 5-MeO-DMT | 6.096 | 138.196 | 99.710 |
| Impurity 1¹ | 7.625 | 0.286 | 0.206 |
| Impurity 2 | Not detected | — | — |
| Impurity 3 | Not detected | — | — |
| Degradation product 1 | 15.084 | 0.055 | 0.039 |
| Degradation product 2 | 16.686 | 0.061 | 0.044 |

¹The amount of Impurity 1 has increased after aerosolization and the additional amount of Impurity 1 is also considered a degradation product.

In conclusion, a highly pure aerosol with only a minimal amount of degradation products, can be generated based on the methods and compositions described herein.

### Example 7. Clinical Evidence for Inhalation of the 5-MeO-DMT Target Dose in a Single Inhalation and for Rapid Systemic Absorption

A clinical trial was performed in which 5-MeO-DMT free base (purity not less than 99%) was administered to patients with treatment-resistant major depressive disorder (TRD). Patients recruited into the trial had to meet the DSM-5 diagnostic criteria for single-episode or recurrent major depressive disorder and had to be treatment-resistant, both aspects as evaluated by a psychiatrist or registered psychologist. On the administration day, a single dose of 12 mg 5-MeO-DMT was administered to the patients via a single inhalation as described in Example 1. Patients were closely monitored for 3.5 hours after administration, with additional follow-up visits 1 day and 7 days after dosing.

Two patients with major depressive disorder were recruited into the study. The inhalation procedure was adequately performed with a single inhalation by both patients and was well tolerated with no inhalation-related adverse events, especially no coughing. The first psychedelic symptoms as assessed by an observer occurred immediately after the inhalation. The psychedelic experience was highly intense with both patients achieving a peak psychedelic experience as assessed by the 30-item revised Mystical Experience Questionnaire (MEQ30) (as described in Barrett FS, J Psychopharmacol. 2015; 29 (11):1182-90). The duration of the psychedelic experience as judged by an external observer was 16 min for patient 1 and 40 minutes for patient 2.

Remarkably, both patients reported a formal remission of their depressive symptoms, as assessed by a score of equal or less than 10 on the Montgomery-Asberg Depression Rating Scale (MADRS), already at the first assessment time point at 2 hours after drug administration, with the effect further deepening at the day 1 and the day 7 follow-up visits.

This data demonstrates that inhalation of an aerosol containing 5-MeO-DMT aerosol particles generated as described in example 1 (i.e., aerosolization of a thin layer of 5-MeO-DMT through flow of air heated to 210° C. at a flow rate of 12 liters/minute for 15 seconds over the thin layer) is well tolerated and can be inhaled within a single inhalation. It also demonstrates that 5-MeO-DMT from such aerosol particles is rapidly systemically absorbed, as evidenced by the rapid onset of psychedelic effects with a few seconds after starting the inhalation. It is considered that such rapid systemic absorption occurs via the pulmonary alveoli.

### Example 8. Evaluation of Mass Median Aerodynamic Diameter of 5-MeO-DMT Aerosols

The particle size distribution of 5-MeO-DMT aerosol particles as generated according to the compositions and methods of the present invention are determined according to United States Pharmacopeia (USP) methods using the Next Generation Impactor (NGI) (United States Pharmacopeia (USP)<601> Apparatus 6) as shown in FIG. 2.

This device fractionates the bolus aerosol delivered into a discrete series of size ranges on the basis of particle or droplet inertia and provides a measure of the aerodynamic diameter of the aerosol droplets produced. The droplet size range captured by each stage in the NGI can be dependent on the measured airflow used. For the present analysis, experiments are carried out using an airflow of 30 liters/minute (Experiments 1 and 3) and of 15 liters/minute (Experiments 2 and 4).

Based on a log-normal distribution of particle sizes, the aerodynamic size distribution is characterized by the mass median aerodynamic diameter (MMAD) and geometric standard deviation (GSD). Moreover, the fine particle fraction (FPF) is determined as the weight percentage of droplets having an aerodynamic diameter of less than or equal to 5 μm, relative to the total of droplets on the impactor.

### Example 8—Experiment 1

A first experiment is carried out using a Volcano Medic Vaporization System as described in Example 1 with dosing capsules having a nominal drug load of 6 mg 5-MeO-DMT free base/capsule. The aerosol recovered in the balloon is measured using the NGI operated using a flow rate of 30 liters/min.

The experiment is run in triplicate.

The results are shown in the table below.

| | MMAD (μm) | GSD | FPF (%) |
|---|---|---|---|
| Run 1 | 0.33 | 4.70 | 96.19 |
| Run 2 | 0.46 | 3.05 | 98.35 |
| Run 3 | 0.42 | 2.71 | 99.31 |
| Mean | 0.40 | 3.49 | 97.95 |

### Example 8—Experiment 2

A second experiment is carried also out using a Volcano Medic Vaporization System as described in Example 1 with dosing capsules having a nominal drug load of 6 mg 5-MeO-DMT free base/capsule. The aerosol recovered in the balloon is measured using the NGI operated using a flow rate of 15 liters/min.

In this experiment, NGI cups were coated with glycerol to prevent possible droplet re-entrainment. In addition, approximately 8 drops of 30% glycerol in water were added to each filter paper located underneath the corresponding NGI Stage Jets.

The experiment is run in triplicate.

The results are shown in the table below.

| | MMAD (μm) | GSD | FPF (%) |
|---|---|---|---|
| Run 1* | 0.17 | 7.49 | 95.22 |
| Run 2 | 0.84 | 3.26 | 93.54 |
| Run 3 | 0.70 | 3.14 | 95.79 |
| Mean (Runs 1-3) | 0.57 | 4.63 | 94.85 |
| Mean (Runs 2, 3) | 0.77 | 3.20 | 94.67 |

*Data may not be reliable due to experimental problems with the cup coating.

### Example 8—Experiment 3

Experiment 1 is repeated using capsules with a nominal drug loading of 18 mg/capsule. The results are shown in the table below.

| | MMAD (μm) | GSD | FPF (%) |
|---|---|---|---|
| Run 1 | 0.98 | 2.98 | 93.13 |
| Run 2 | 1.19 | 2.52 | 94.08 |
| Run 3 | 0.97 | 2.44 | 96.72 |
| Mean | 1.05 | 2.65 | 94.64 |

### Example 8—Experiment 4

Experiment 2 is repeated using capsules with a nominal drug loading of 18 mg/capsule. The results are shown in the table below.

| | MMAD (μm) | GSD | FPF (%) |
|---|---|---|---|
| Run 1 | 1.16 | 2.66 | 93.23 |
| Run 2 | 1.14 | 2.77 | 92.66 |
| Run 3 | 1.82 | 2.10 | 91.40 |
| Mean | 1.37 | 2.51 | 92.43 |

The above data show that the MMAD is less than 3 μm and more than 0.1 μm, in particular less than 2.5 μm and more than 0.1 μm, especially less than 2 μm and more than 0.1 μm. At least 80 wt %, in particular at least 85 wt % and especially at least 90 wt % of the aerosol particles (droplets) have an aerodynamic diameter of less than or equal to 5 μm.

### Example 9. Delivered Dose Determination-Prophetic Example

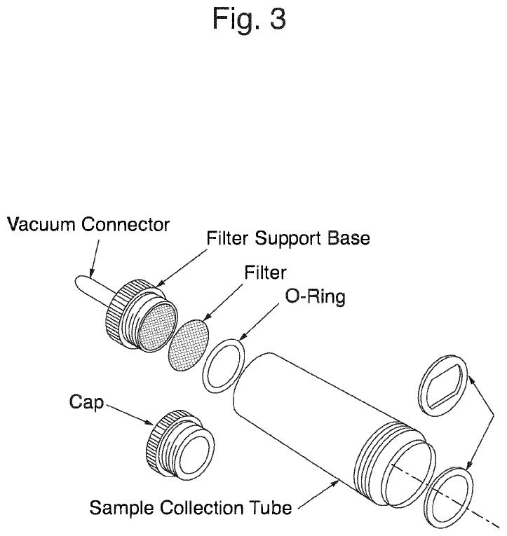

The 5-MeO-DMT amount delivered to the patient by the compositions and methods according to the invention are confirmed according to United States Pharmacopeia (USP) methods by collection of the aerosol in a suitable aerosol sampling apparatus such as United States USP <601> Apparatus A shown in FIG. 3. The aerosol is typically sampled at a flow rate of 28.3 Liters/minute. Once aerosol sampling is complete both ends of the dose tube are capped and the drug is extracted from the filter using a suitable recovery solvent and assayed using a suitably validated analytical technique.

### Example 10. Preparation of Starting Material

5-MeO-DMT (2.0 g) was dissolved in MTBE (4 mL, 2.0 volumes) at 35 to 40° C. before being cooled to room temperature over 30 minutes. After stirring at room temperature for 50 minutes no crystallisation was observed, therefore, the batch temperature was decreased to 7 to 12° C. over 30 minutes. After stirring at 7 to 12° C. for 10 minutes crystallisation occurred. The batch was subsequently filtered following a 1 hour stir out at 7 to 12° C. After washing with MTBE (1 mL, 0.5 volumes), at 7 to 12° C., the batch was pulled dry under vacuum for 3.5 hours to yield a pale orange solid in 1.02 g (50% recovery). The isolated solid was analysed for purity by HPLC. The purity was found to be 99.74% area.

The table below displays the impurity profile of isolated material.

### Example 10, Table 1. Impurity Profile of Isolated Material

| Impurity Profile | | HPLC Purity (area %) | |
|---|---|---|---|
| | RRT | Raw Material | Isolated Material |
| | 0.87 | 0.07 | 0.06 |
| | 0.90 | 0.04 | 0.02 |
| | 0.92 | 0.03 | — |
| 5-MeO-DMT | 1.00 | 99.21 | 99.74 |
| | 1.18 | 0.13 | 0.04 |
| | 1.24 | 0.15 | 0.02 |
| | 1.28 | 0.02 | <0.01 |
| | 1.64 | — | 0.02 |
| | 1.67 | — | <0.01 |
| | 1.72 | — | — |
| | 1.96 | 0.02 | — |
| | 2.08 | — | — |
| | 2.11 | — | — |
| | 2.34 | 0.03 | — |
| | 2.38 | 0.29 | 0.08 |
| | 2.42 | — | — |
| | 2.61 | — | — |
| | 2.76 | 0.01 | — |
| | 2.82 | — | — |
| | 2.90 | — | — |

The results from the analysis indicated that the overall purity of the material was increased and the impurities at RRT 1.18 and at RRT 1.24 were purged to below 0.10%. The impurity at RRT 2.38 was also reduced to below the target of NMT 0.10%.

Solvent analysis of sample indicated an MTBE level of 17 ppm against an expected limit of NMT 5000 ppm.

1. A pharmaceutical composition comprising:
an aerosol comprising a therapeutically effective amount of aerosol particles of 5-methoxy-N,N-dimethyltryptamine (5-MeO-DMT) and/or a pharmaceutically acceptable salt thereof in a pharmaceutically acceptable gas,
wherein the aerosol particles have a mass median aerodynamic diameter (MMAD) of 0.1 μm to 3 μm, and
wherein the aerosol is contained in a volume for single inhalation by a patient.

2. The pharmaceutical composition of claim 1, wherein the aerosol particles have a fine particle fraction (FPF), determined as a weight percentage of aerosol particles with an aerodynamic diameter of less than or equal to 5 μm relative to a total mass of the aerosol particles, of at least 90 wt %.

3. The pharmaceutical composition of claim 2, wherein the FPF and MMAD are determined with a Next Generation Impactor (NGI) in accordance with methods shown in United States Pharmacopeia (USP)<601>.

4. The pharmaceutical composition of claim 3, wherein the NGI is operated at an airflow of 15 liters/minute or 30 liters/minute.

5. The pharmaceutical composition of claim 1, wherein the MMAD is determined with a Next Generation Impactor (NGI) in accordance with methods shown in United States Pharmacopeia (USP) <601>.

6. The pharmaceutical composition of claim 5, wherein the NGI is operated at an airflow of 15 liters/minute or 30 liters/minute.

7. The pharmaceutical composition of claim 1, wherein the pharmaceutically acceptable gas is air.

8. The pharmaceutical composition of claim 1, wherein the aerosol is contained in a volume of less than 3 liters.

9. The pharmaceutical composition of claim 1, wherein the aerosol is contained in a volume of about 1.5 to 2 liters.

10. The pharmaceutical composition of claim 1, wherein the aerosol is contained in a volume of about 2 liters.

11. The pharmaceutical composition of claim 1, wherein the aerosol particles comprise 5-methoxy-N,N-dimethyltryptamine freebase.

12. The pharmaceutical composition of claim 1, wherein the aerosol particles comprise a pharmaceutically acceptable salt of 5-methoxy-N,N-dimethyltryptamine.

13. The pharmaceutical composition of claim 1, wherein the aerosol has an aerosol particle mass density of about 0.5 mg/L to about 12.5 mg/L.

14. The pharmaceutical composition of claim 1, wherein the aerosol has an aerosol particle mass density of about 1 mg/L to about 10 mg/L.

15. A method of treating major depressive disorder (MDD), the method comprising:
administering to a patient diagnosed with major depressive disorder a pharmaceutical aerosol comprising a therapeutically effective amount of aerosol particles of 5-methoxy-N,N-dimethyltryptamine (5-MeO-DMT) and/or a pharmaceutically acceptable salt thereof,
wherein the administration achieves clinical remission of the major depressive disorder.

16. The method of claim 15, wherein the patient suffers from a treatment resistant form of major depressive disorder (TRD).

17. The method of claim 15, wherein the clinical remission is assessed by a score of equal to or less than 10 on the Montgomery-Asberg Depression Rating Scale (MADRS).

18. The method of claim 15, wherein clinical remission is achieved by at least 7 days from administration of the pharmaceutical aerosol.

19. The method of claim 15, wherein clinical remission is achieved by at least 1 day from administration of the pharmaceutical aerosol.

20. The method of claim 15, wherein the administration of the pharmaceutical aerosol is by inhalation.

21. The method of claim 20, wherein the administration of the pharmaceutical aerosol is by a single inhalation.

22. The method of claim 15, wherein the aerosol particles have a mass median aerodynamic diameter (MMAD) of 0.1 μm to 3 μm.

23. The method of claim 22, wherein the MMAD is determined with a Next Generation Impactor (NGI) in accordance with methods shown in United States Pharmacopeia (USP)<601>.

24. The method of claim 15, wherein the aerosol particles have a fine particle fraction (FPF), determined as the weight percentage of aerosol particles having an aerodynamic diameter of less than or equal to 5 μm relative to the total of mass of the aerosol particles, of at least 90 wt %.

25. The method of claim 24, wherein the FPF is determined with a Next Generation Impactor (NGI) in accordance with methods shown in United States Pharmacopeia (USP)<601>.

26. The method of claim 15, wherein the pharmaceutical aerosol is contained in a volume of less than 3 liters.

27. The method of claim 15, wherein the pharmaceutical aerosol is contained in a volume of about 1.5 to 2 liters.

28. The method of claim 15, wherein the pharmaceutical aerosol is contained in a volume of about 2 liters.

29. The method of claim 15, wherein the aerosol particles comprise 5-methoxy-N,N-dimethyltryptamine freebase.

30. The method of claim 15, wherein the aerosol particles comprise a pharmaceutically acceptable salt of 5-methoxy-N,N-dimethyltryptamine.
